(19) United States
(12) Patent Application Publication
(10) Pub. No.: US 2021/0363690 A1
(43) Pub. Date: Nov. 25, 2021

(54) Fabrics with Improved Barrier Properties

(71) Applicant: Berry Global, Inc., Evansville, IN (US)

(72) Inventors: Lei Wang, Mooresville, NC (US); Nyle Bishop, Mooresville, NC (US); Fernando Marin, Huntersville, NC (US)

(21) Appl. No.: 17/324,155

(22) Filed: May 19, 2021

Related U.S. Application Data
(60) Provisional application No. 63/026,921, filed on May 19, 2020.

(51) Int. Cl. D06M 15/277; D06M 10/02; D06M 15/256; D04H 3/013; D04H 3/007; D04H 3/11

(52) U.S. Cl. CPC D06M 15/277; D06M 10/025; D06M 15/256; D06M 2101/20; D04H 3/007; D04H 3/11; D06M 2200/10; D04H 3/013

(57) ABSTRACT

A fabric suitable as an alcohol repellent fabric is provided. The fabric includes a fibrous substrate including a first outermost surface and a second outermost surface, in which a digitally printed or sprayed alcohol repellent composition is located on at least a portion the first outermost surface, at least a portion of the second outermost surface, or both. The fabric also includes an antistatic composition located on at least a portion the first outermost surface, at least a portion of the second outermost surface, or both.

### CROSS-REFERENCE TO RELATED APPLICATIONS

This application claims priority under 35 U.S.C. § 119(e) to U.S. Provisional Application No. 63/026,921 filed May 19, 2020, which is expressly incorporated by reference herein in its entirety.

### TECHNICAL FIELD

Embodiments of the presently-disclosed invention relate generally to fabrics suitable as an alcohol repellent fabric, in which the fabric includes a digitally printed or sprayed alcohol repellent composition on at least a portion of a first outermost surface of the fabric, at least a portion of a second outermost surface of the fabric, or both. The fabric may also include an antistatic composition located on at least a portion the first outermost surface, at least a portion of the second outermost surface, or both.

### BACKGROUND

Alcohol repellent fabrics are frequently used in surgical drapes and gowns. These fabrics often consist of barrier fabrics treated with fluorochemicals to enhance resistance to penetration by isopropyl alcohol. Until recently, C8 fluorochemicals were commonly used in such applications, but there has been a shift toward treating fabrics with C6 fluorochemicals instead. However, because C6 fluorochemicals have fewer fluorinated carbons, the fabric must be treated with a higher level of C6 fluorochemicals to achieve the same alcohol repellency achieved by the C8 fluorochemicals. Since C6 fluorochemicals are expensive, the cost of alcohol repellent fabric has increased tremendously.

In addition to providing resistance to alcohol repellency, alcohol repellent fabrics when used in surgical drapes and gowns must also provide the necessary dissipation level or rate of static electricity is important to prevent a build-up of charges that may affect the ability of the gown to drape naturally stay in place. For instance, a lack of charge dissipation may cause the gown or drape to stick to the body and the slowly creep up with the movements of the use.

Alcohol repellent fabrics are produced with traditional topical padding methods, such as a “dip/nip with can or oven drying/curing” of the repellent composition and the antistatic composition or finish. The traditional approach consumes significant amount of water and energy in the treatment process and requires undesirable and costly waste water cleanup. Typically, the amount of water used is about 100% to about 120% of the weight of the nonwoven fabric when using a traditional topical padding process for the alcohol repellent composition and the antistatic composition or finish. Accordingly, such processes requires from about 1,000 to about 1,200 kg of water when 1,000 kg of fabric is treated to provide an alcohol repellent fabric. Moreover, the 1,000 to 1,200 kg of water will ultimately need to be evaporated into the atmosphere after and/or during the thermal drying process. The post heat thermal degradation (material tensile strength and tear strength loss) associated with such processes may become readily apparent during storage.

Therefore, there at least remains a need in the art for an alcohol repellent fabric that provides alcohol repellency and static dissipation. There also remains a need in the art for a process for producing an alcohol repellent fabric that reduces or eliminates the production of waste water associated with coating the fabric with an alcohol repellent composition.

### SUMMARY OF INVENTION

One or more embodiments of the invention may address one or more of the aforementioned problems. Certain embodiments according to the invention provide a fabric comprising a fibrous substrate including a first outermost surface and a second outermost surface. The fabric may comprise a digitally printed or sprayed alcohol repellent composition on at least a portion of a first outermost surface of the fabric, at least a portion of a second outermost surface of the fabric, or both. The fabric may also include an antistatic composition located on at least a portion the first outermost surface, at least a portion of the second outermost surface, or both.

In another aspect, the present invention provides a method of forming a fabric. The method may comprise providing a fibrous substrate including a first outermost surface and a second outermost surface and digitally printing or spraying an alcohol repellent composition on at least a portion of a first outermost surface of the fabric, at least a portion of a second outermost surface of the fabric, or both. The method may also optionally comprise applying an antistatic composition on at least a portion of a first outermost surface of the fabric, at least a portion of a second outermost surface of the fabric, or both. The method may also comprise curing the alcohol repellent composition to provide an alcohol repellent fabric.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates a schematic for forming SMMMS fabrics according to certain embodiments of the invention; and

FIG. 2 illustrates a schematic of coating and curing operation according to certain embodiments of the invention.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The presently-disclosed invention relates generally to the terms “substantial” or “substantially” may encompass the whole amount as specified, according to certain embodiments of the invention, or largely but not the whole amount specified (e.g., 95%, 96%, 97%, 98%, or 99% of the whole amount specified) according to other embodiments of the invention.

The terms “polymer” or “polymeric”, as used interchangeably herein, may comprise homopolymers, copolymers, such as, for example, block, graft, random, and alternating copolymers, terpolymers, etc., and blends and modifications thereof. Furthermore, unless otherwise specifically limited, the term “polymer” or “polymeric” shall include all possible structural isomers; stereoisomers including, without limitation, geometric isomers, optical isomers or enantiomers; and/or any chiral molecular configuration of such polymer or polymeric material. These configurations include, but are not limited to, isotactic, syndiotactic, and atactic configurations of such polymer or polymeric material. The term “polymer” or “polymeric” shall also include polymers made from various catalyst systems including, without limitation, the Ziegler-Natta catalyst system and the metallocene/single-site catalyst system. The term “polymer” or “polymeric” shall also include, in according to certain embodiments of the invention, polymers produced by fermentation process or biosourced.

The terms “nonwoven” and “nonwoven web”, as used herein, may comprise a web having a structure of individual fibers, filaments, and/or threads that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process.

The term “layer”, as used herein, may comprise a generally recognizable combination of similar material types and/or functions existing in the X-Y plane.

The term “spunbond”, as used herein, may comprise fibers which are formed by extruding molten thermoplastic material as filaments from a plurality of fine, usually circular, capillaries of a spinneret with the diameter of the extruded filaments then being rapidly reduced. According to an embodiment of the invention, spunbond fibers are generally not tacky when they are deposited onto a collecting surface and may be generally continuous as disclosed and described herein. It is noted that the spunbond used in certain composites of the invention may include a nonwoven described in the literature as SPINLACE®.

As used herein, the term “continuous fibers” refers to fibers which are not cut from their original length prior to being formed into a nonwoven web or nonwoven fabric. Continuous fibers may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

The term “melt fibrillation”, as used herein, may comprise a general class of making fibers defined in that one or more polymers are molten and may be extruded into many possible configurations (e.g. co-extrusion, homogeneous or bicomponent films or filaments) and then fibrillated or fiberized into a plurality of individual filaments for the formation of melt-fibrillated fibers. Non limiting examples of melt-fibrillation methods may include melt blowing, melt fiber bursting, and melt film fibrillation. The term “melt-film fibrillation”, as used herein, may comprise a method in which a melt film is produced from a melt and then a fluid is used to form fibers (e.g., melt-film fibrillated fibers) from the melt film. Examples include U.S. Pat. Nos. 6,315,806, 5,183,670, 4,536,361, 6,382,526, 6,520,425, and 6,695,992, in which the contents of each are incorporated by reference herein to the extent that such disclosures are consistent with the present disclosure. Additional examples include U.S. Pat. Nos. 7,628,941, 7,722,347, 7,666,343, 7,931,457, 8,512,626, and 8,962,501, which describe the Arium™ melt-film fibrillation process for producing melt-film fibrillated fibers (e.g., having sub-micron fibers).

As used herein, the term “aspect ratio”, comprise a ratio of the length of the major axis to the length of the minor axis of the cross-section of the fiber in question.

The term “multi-component fibers”, as used herein, may comprise fibers formed from at least two different polymeric materials or compositions (e.g., two or more) extruded from separate extruders but spun together to form one fiber. The term “bi-component fibers”, as used herein, may comprise fibers formed from two different polymeric materials or compositions extruded from separate extruders but spun together to form one fiber. The polymeric materials or polymers are arranged in a substantially constant position in distinct zones across the cross-section of the multi-component fibers and extend continuously along the length of the multi-component fibers. The configuration of such a multi-component fiber may be, for example, a sheath/core arrangement wherein one polymer is surrounded by another, an eccentric sheath/core arrangement, a side-by-side arrangement, a pie arrangement, or an “islands-in-the-sea” arrangement, each as is known in the art of multicomponent, including bicomponent, fibers.

The term “fluorochemical”, as used herein, may comprise any of various chemical compounds containing fluorine, particularly organic compounds (e.g., fluorocarbons such as perfluoroalkanes) in which fluorine has replaced a large proportion of the hydrogen attached to the carbons. Fluorochemicals may exhibit low surface tension and low viscosity and are extremely stable due to the strength of the carbon-fluorine bond. Fluorochemicals are not miscible with most organic solvents.

The term “dry basis”, as used herein may comprise the calculation or measurement of a weight percentage in which the presence of water and/or other solvents (e.g., alcohols) are ignored or excluded for purposes of the calculation or measurement. Weight percentages may frequently be measured on a dry basis to remove the effects of evaporation and/or condensation which may happen naturally throughout the useful life of a composition or article.

The term “cellulosic fiber”, as used herein, may comprise fibers derived from hardwood trees, softwood trees, or a combination of hardwood and softwood trees prepared for use in, for example, a papermaking furnish and/or fluff pulp furnish by any known suitable digestion, refining, and bleaching operations. The cellulosic fibers may comprise recycled fibers and/or virgin fibers. Recycled fibers differ from virgin fibers in that the fibers have gone through the drying process at least once. In certain embodiments, at least a portion of the cellulosic fibers may be provided from non-woody herbaceous plants including, but not limited to, kenaf, cotton, hemp, jute, flax, sisal, or abaca. Cellulosic fibers may, in certain embodiments of the invention, comprise either bleached or unbleached pulp fiber such as high yield pulps and/or mechanical pulps such as thermo-mechanical pulping (TMP), chemical-mechanical pulp (CMP), and bleached chemical-thermo-mechanical pulp BCTMP. In this regard, the term “pulp”, as used herein, may comprise cellulose that has been subjected to processing treatments, such as thermal, chemical, and/or mechanical treatments. Cellulosic fibers, according to certain embodiments of the invention, may comprise one or more pulp materials.

All whole number end points disclosed herein that can create a smaller range within a given range disclosed herein are within the scope of certain embodiments of the invention. By way of example, a disclosure of from about 10 to about 15 includes the disclosure of intermediate ranges, for example, of: from about 10 to about 11; from about 10 to about 12; from about 13 to about 15; from about 14 to about 15; etc. Moreover, all single decimal (e.g., numbers reported to the nearest tenth) end points that can create a smaller range within a given range disclosed herein are within the scope of certain embodiments of the invention. By way of example, a disclosure of from about 1.5 to about 2.0 includes the disclosure of intermediate ranges, for example, of: from about 1.5 to about 1.6; from about 1.5 to about 1.7; from about 1.7 to about 1.8; etc.

Certain embodiments according to the invention generally relate to alcohol repellent fabrics having a digitally printed or sprayed alcohol repellent composition provided thereon. An alcohol repellent composition in a pre-cured form may be digitally printed or sprayed onto a fibrous substrate and subsequently cured (e.g., radically cured). In accordance with certain embodiments of the invention, the pre-cured alcohol repellent composition may be selectively applied to one or more areas of the fibrous substrate via stencil printing, screen printing, flexographic printing, laser printing, inkjet printing, extrusion 3D printing, or any combination thereof. In accordance with certain embodiments of the invention, the curing operation may comprise subjecting the pre-cured alcohol repellent composition to one or more atmospheric-pressure plasma operations to form a cured alcohol repellent composition or coating that may be adhered and/or bonded to the fibrous substrate. In accordance with certain embodiments of the invention, the cured alcohol repellent composition may be located on merely a portion or portions (or the entirety) of one side of the fibrous substrate, respective portions of both sides of the fibrous substrate, or the entirety of both sides of the fibrous substrate. Similarly, an antistatic composition may be deposited on one or both sides of the fibrous substrate. For example, the fibrous substrate may comprise an alcohol repellent composition on one or more portions of a first side of the fibrous substrate and the antistatic composition located on one or more portions of a second side of the fibrous substrate. In accordance with certain embodiments, both sides of the fibrous substrate may comprise an alcohol repellent composition and an antistatic agent. In accordance with certain embodiments of the invention, the alcohol repellent composition and the antistatic composition may be either overlaid or formulated, applied, and cured together as a single composition.

In accordance with certain embodiments of the invention, a method of digitally printing or spraying the alcohol repellent composition and curing of this composition, for example, by one or more atmospheric-pressure plasma operation may significantly reduce and/or provide a treatment process that is substantially devoid of water usage and/or generation of wastewater. Moreover, such a process may be a non-thermal process (e.g., the fibrous substrate does not or is not subjected to external heat for curing and/or drying of the alcohol repellent composition deposited thereon), which mitigates or eliminates thermal degradation of the resulting alcohol repellent fabric, reduces energy consumption, and substantially (or completely) eliminates the creation of greenhouse gases associated with these processing activities (e.g., coating and curing of an alcohol repellent composition onto a fibrous substrate).

In accordance with certain embodiments of the invention, the alcohol repellent composition includes a fluorochemical compound or compounds. While in a pre-cured state, the alcohol repellent composition may include one or monomers that that include one or more fluorochemical chains and one or more polymerizable functional groups (e.g., free-radically polymerizable functional groups that polymerize by the action of free radicals). For instance, the one or more fluorochemical chains may impart the desired alcohol repellency in the cured composition, while the one or more polymerizable functional groups enable, for example, a radically-mediated curing of the pre-cured alcohol repellent composition, such as upon exposure to one or more atmospheric plasma curing operations. Although the polymerizable functional groups are not particularly limited, acrylate and/or methacrylate groups among other groups having double and/or triple bonds may be used in accordance with certain embodiments of the invention. For instance, the polymerizable functional groups may comprise an acrylate or methacrylate group, an allylic group, an alkyne group, a styrenic group, a vinyl ether group, a vinyl ester group, a vinyl amide group, a maleate group, a fumarate group, a crotonate group, a cinnamate group, or a norbornene group. After being applied to the fibrous structure, the pre-cured alcohol repellent composition may be moved into the vicinity of plasma treatment regions such that excited species therefrom impinge thereon. The polymerizable monomers cure upon exposure to the plasma treatment forming a cured alcohol repellent composition that adheres to the fibrous structure. As an example, the hydrocarbon portions of polymerized 2-(Perfluorohexyl) ethyl acrylate (commonly referred to as C6) bond to each other and to the fibrous structure, while the fluorinated chains face away from the fibrous structure and repel alcohol, water, and/or oil. In accordance with certain embodiments of the invention, the pre-cured and/or cured alcohol composition comprises less than about 5% by weight of water, less than about 3% by weight of water, less than about 2% by weight of water, or less than about 1% by weight of water, or less than about 0.5% by weight of water. In accordance with certain embodiments of the invention, the pre-cured and/or cured alcohol composition is devoid of water.

Certain embodiments according to the invention provide a fabric comprising a fibrous substrate including a first outermost surface and a second outermost surface. The fabric may comprise a digitally printed or sprayed alcohol repellent composition on at least a portion of a first outermost surface of the fabric, at least a portion of a second outermost surface of the fabric, or both. The fabric may also include an antistatic composition located on at least a portion the first outermost surface, at least a portion of the second outermost surface, or both. For example, the digitally printed or sprayed alcohol repellent composition may be located on at least a portion of the first outermost surface and also on at least the second outermost surface. The first outermost surface may have the digitally or sprayed alcohol repellent composition on one or more separate and discrete locations or, alternatively, be completely coated with the digitally or sprayed alcohol repellent composition. In accordance with certain embodiments of the invention, the second outermost surface may have the digitally or sprayed alcohol repellent composition on one or more separate and discrete locations or, alternatively, be completely coated with the digitally or sprayed alcohol repellent composition.

In accordance with certain embodiments of the invention, the digitally printed or sprayed alcohol repellent composition may be located on at least the first outermost surface and located in one or more treated discrete area. For example, the one or more discrete treated areas may cover from about 1% to about 99% of the first outermost surface, such as at least about any of the following: 1, 3, 5, 10, 15, 20, 25, 30, 35, 40, 45, and 50% and/or at most about any of the following: 99, 95, 90, 85, 80, 75, 70, 65, 60, 55, and 50%. Additionally or alternatively, the second outermost surface may comprise digitally printed or sprayed alcohol repellent composition located in one or more treated discrete area of the second outermost surface. For example, the one or more discrete treated areas of the second outermost surface may cover from about 1% to about 99% of the second outermost surface, such as at least about any of the following: 1, 3, 5, 10, 15, 20, 25, 30, 35, 40, 45, and 50% and/or at most about any of the following: 99, 95, 90, 85, 80, 75, 70, 65, 60, 55, and 50%.

In accordance with certain embodiments of the invention, the antistatic composition may be located on at least a portion of the first outermost surface and also on at least the second outermost surface. The first outermost surface may have the antistatic composition on one or more separate and discrete locations or, alternatively, be completely coated with the antistatic composition. In accordance with certain embodiments of the invention, the second outermost surface may have the antistatic composition on one or more separate and discrete locations or, alternatively, be completely coated with the antistatic composition. As noted above, the digitally or sprayed alcohol repellent composition and the antistatic composition may be applied separately to the fibrous substrate and/or applied to different regions of the fibrous substrate, whether on the same side of the fibrous substrate or the same side of the fibrous substrate. Additionally or alternatively, the digitally or sprayed alcohol repellent composition and the antistatic composition may be formulated as a single composition including the constituents of both the digitally or sprayed alcohol repellent composition and the antistatic composition. In this regard, the single composition may provide a method of simultaneously applying a fluorochemical and an antistatic agent.

In accordance with certain embodiments of the invention, the digitally printed or sprayed alcohol repellent composition comprises a plasma cured composition including at least one fluorochemical comprising at least one C4 fluorochemical, a C6 fluorochemical, a C8 fluorochemical, a C10 fluorochemical, or any combination thereof. For example, the digitally printed or sprayed alcohol repellent composition may comprise a plasma cured network formed by the reaction product of one or more monomers, in which the one more monomers have one or more fluorochemical chains and one or more polymerizable functional groups (e.g., free-radically polymerizable functional groups that polymerize by the action of free radicals) as noted above. For instance, the one or more monomers have polymerized upon exposure to a plasma treatment (e.g., atmospheric plasma treatment) via the one or more polymerizable functional groups. Stated somewhat differently, the digitally printed or sprayed alcohol repellent composition may comprise a plasma cured composition, in which the plasma cured composition comprises a polymerized composition of a free-radically curable monomeric composition including at least a first group of monomers including at least one free-radically polymerizable functional group and at least one side chain comprising from about 4 to about 10 fluorinated carbon atoms, such as 4, 5, 6, 7, 8, 9, or 10 fluorinated carbon atoms. For example, the digitally printed or sprayed alcohol repellent composition may comprise a plasma cured composition, in which the plasma cured composition comprises a polymerized composition including a perfluoroalkylethylmethacrylate containing at least one side chain from about 4 to about 10 fluorinated carbon atoms, such as 4, 5, 6, 7, 8, 9, or 10 fluorinated carbon atoms.

In accordance with certain embodiments of the invention, the digitally printed or sprayed alcohol repellent composition may comprise from about 0.01 to about 10% by weight of the fabric, such as at least about 0.01, 0.05, 0.1, 0.25, 0.5, 0.75, 1, 1.5, 2, 2.5, 3, 3.5, 4, 4.5, and 5% by weight of the fabric and/or at most about any of the following: 10, 9, 8, 7, 6, and 5% by weight of the fabric.

In accordance with certain embodiments of the invention, the fabric may comprise from about 0.01 to about 1% by weight of fluorine as determined by ASTM D240, such as at least about 0.01, 0.05, 0.1, 0.125, 0.15, 0.175, 0.2, 0.225, 0.25, 0.275, 0.3, 0.35, 0.4, and 0.5% by weight of fluorine as determined by ASTM D240 and/or at most about any of the following: 1, 0.75, 0.5, and 0.4% by weight of fluorine as determined by ASTM D240. In accordance with certain embodiments of the invention, the weight percentages of fluorine enumerated above is attributed completely from the digitally printed or sprayed alcohol repellent composition.

In accordance with certain embodiments of the invention, the antistatic composition comprises at least one antistatic agent, in which the antistatic composition comprises at least one of a non-ionic antistatic agent, an anionic antistatic agent, a cationic antistatic agent, an amphoteric antistatic agent, or any combination thereof. In accordance with certain embodiments of the invention, the at least one antistatic agent comprises an alkylphosphate or a phosphate ester.

In accordance with certain embodiments of the invention, the antistatic composition may comprise from about 0.01 to about 10% by weight of the fabric, such as at least about 0.01, 0.05, 0.1, 0.25, 0.5, 0.75, 1, 1.5, 2, 2.5, 3, 3.5, 4, 4.5, and 5% by weight of the fabric and/or at most about any of the following: 10, 9, 8, 7, 6, and 5% by weight of the fabric.

In accordance with certain embodiments of the invention, the fabric has a first ratio between the digitally printed or sprayed alcohol repellent composition (% weight of the fabric on a dry basis) to the antistatic composition (% weight of the fabric on a dry basis) from about 0.2:1 to about 3:1, such as at least about any of the following: 0.2:1, 0.4:1, 0.6:1, 0.8:1, 1:1 and/or at most about any of the following: 3:1, 2.5:1, 2:1, 1.5:1, and 1:1.

In accordance with certain embodiments of the invention, the fabric has a second ratio between the fluorine content attributed to the digitally printed or sprayed alcohol repellent composition (% weight of the fabric on a dry basis) to the antistatic agent (% weight of the fabric on a dry basis) from about 0.2:1 to about 3:1, such as at least about any of the following: 0.2:1, 0.4:1, 0.6:1, 0.8:1, 1:1 and/or at most about any of the following: 3:1, 2.5:1, 2:1, 1.5:1, and 1:1.

In accordance with certain embodiments of the invention, the first outermost surface, the second outermost surface, or both may comprise as noted above a blended coating in cured form including the digitally printed or sprayed alcohol repellent composition and that antistatic composition.

The fibrous substrate, in accordance with certain embodiments of the invention, may have a basis weight from about 5 to about 200 grams-per-square meter (gsm), such as at least about any of the following: 5, 8, 10, 12, 15, 18, 20, 30, 40, 50, 60, 70, 80, 90, and 100 gsm and/or at most about any of the following: 200, 175, 150, 125, and 100 gsm.

In accordance with certain embodiments of the invention, the fabric may comprise a third ratio between the digitally printed or sprayed alcohol repellent composition (% weight of the fabric on a dry basis) to the basis weight (gsm) of the fabric from about 1:10,000 to about 1:10, such as at least about any of the following: 1:10,000, 1:8000, 1:6000, 1:4000, 1:2000, and 1:1000 and/or at most about any of the following: 1:10, 1:25, 1:50, 1:75, 1:100, and 1:1000.

In accordance with certain embodiments of the invention, the fabric may comprise a fourth ratio between the fluorine content attributed to the digitally printed or sprayed alcohol repellent composition (% weight of the fabric on a dry basis) to the basis weight (gsm) of the fabric from about 1:10,000 to about 1:10, such as at least about any of the following: 1:10,000, 1:8000, 1:6000, 1:4000, 1:2000, and 1:1000 and/or at most about any of the following: 1:10, 1:25, 1:50, 1:75, 1:100, and 1:1000.

In accordance with certain embodiments of the invention, the fibrous substrate may comprise one or more woven materials, one or more nonwoven materials, one or more film layers, one or more natural and/or synthetic cellulose layers (e.g., pulp, paper, tissue, etc.), or any combination thereof. In accordance with certain embodiments of the invention, the one or more nonwoven materials may comprise one or more spunbond layers, one or more meltblown layers, one or more melt-fibrillated layers, one or more electrospun layers, one or more carded nonwoven layers, one or more hydroentangled layers, or any combinations thereof. For example, the fibrous substrate may comprise a SMS, S, SS, SSS, Meltblown itself or hydroentangled fiber or pulp alone or in combination with any of the nonwoven layer or layers described and disclosed herein.

In accordance with certain embodiments of the invention, the fibrous substrate may comprise both cellulosic and synthetic fibers. For example, the fibrous substrate may comprise one or more physically entangled (e.g., hydroentangled) nonwoven layers comprising synthetic fibers alone or in combination with cellulosic fibers (e.g., pulp, rayon, viscose, etc.) The fibrous substrate, for instance, may include a physically entangled (e.g., hydroentangled) nonwoven layer including continuous spunbond fibers, which may comprise a polyolefin such as a polypropylene, and cellulosic fibers or pulp. By way of example only, one or more spunbond layers and one or more cellulosic layer (e.g., air-laid pulp layer, tissue layer, etc.) may be stacked and subjected to hydroentanglement to physically consolidate the spunbond and the cellulosic fibers into a single nonwoven layer.

In accordance with certain embodiments of the invention, the fibrous structure may comprise one of the following structures:

S1ₐ-M_{b}-S2_{c}; (Structure 1)

S1ₐ-N_{d}-S2_{c}; (Structure 2)

S1ₐ-M_{b}-N_{d}-S2_{c}; (Structure 3)

S1ₐ-N_{d}-S3ₑ-N_{d}-S2_{c}; (Structure 4)

S1ₐ-N_{d}-M_{b}-N_{d}-S2_{c}; (Structure 5)

S1ₐ-M_{b}-S3ₑ-M_{b}-S2_{c}; (Structure 6)

S1ₐ-M_{b}-N_{b}-M_{b}-S2_{c}; or any combinations thereof; (Structure 7)

wherein

‘M’ comprises a meltblown layer;

‘N’ comprises a sub-micron fiber-containing layer;

‘S1’ comprises a first spunbond layer;

‘S2’ comprises a second spunbond layer;

‘S3’ comprises a third spunbond layer;

a′ represents the number of layers and is independently selected from 1, 2, 3, 4, and 5;

‘b’ represents the number of layers is independently selected from 1, 2, 3, 4, and 5;

‘c’ represents the number of layers is independently selected from 1, 2, 3, 4, and 5; and

‘d’ represents the number of layers is independently selected from 1, 2, 3, 4, and 5;

‘e’ represents the number of layers is independently selected from 1, 2, 3, 4, and 5.

The fibrous structure, in accordance with certain embodiments of the invention, may comprise one or more layers containing a plurality of cellulosic fibers, in which the plurality of cellulosic fibers comprises a plurality of natural synthetic fibers, a plurality of synthetic cellulosic fibers, or combinations thereof. In accordance with certain embodiments of the invention, the plurality of cellulosic fibers may be physically entangled with a plurality of spunbond fibers, a plurality of meltblown fibers, a plurality of staple fibers, or any combination thereof. As noted above, the fibrous structure may be physically entangled (e.g., hydroentangled) with a variety of layers, including any fibrous structures according to Structures 1-7.

In accordance with certain embodiments of the invention, the fibrous structure may comprise from about 0 to about 60% by weight of meltblown fibers, such as at least about any of the following: 0, 5, 10, 15, 20, 25, 30, and 35% by weight and/or at most about any of the following: 60, 55, 50, 45, 40, and 35% by weight.

In accordance with certain embodiments of the invention, the fabric may comprise at least one binder. For example, the at least one binder may comprise at least one of an acrylic binder, a styrene-butadiene rubber binder, a vinyl copolymer binder, a vinyl acetate binder, an ethylene vinyl acetate binder, a polyvinyl chloride binder, a polyurethane binder, or any combination thereof. In accordance with certain embodiments of the invention, the at least one binder comprises an acrylic binder, such as an anionic acrylic binder, a cationic acrylic binder, or a non-ionic acrylic binder. In accordance with certain embodiments of the invention, the fabric may be devoid of a binder.

In accordance with certain embodiments of the invention, the fibrous substrate comprises one or more spunbond layers, one or more meltblown layers, one or more melt-fibrillated layers, one or more electrospun layers, one or more carded nonwoven layers, and/or the one or more hydroentangled layers that may be independently from each other comprise a synthetic polymer, such as such as a polyolefin, a polyester, a polyamide, or any combination thereof. The polyolefin, for example, may comprise a polypropylene, a polypropylene copolymer, a polyethylene, a polyethylene copolymer, or any combination thereof.

In accordance with certain embodiments of the invention, the fabric has an alcohol repellency rating of at least 7 as determined according to IST 80.8, or at least about 8 as determined according to IST 80.8.

In accordance with certain embodiments of the invention, the fabric has a static decay of from about 0.01 to about 0.5 seconds as tested according to IST 40.2 performed at 50% R.H. and using 10% remaining charge as the cut-off level, such as at least about any of the following: 0.01, 0.02, 0.05, 0.08, and 0.1 seconds and/or at most about any of the following: 5, 4, 3, 2, 1.5, 1.2, and 1 seconds.

In accordance with certain embodiments of the invention, the fabric has a static decay of from about 1 to about 3 seconds as tested according to IST 40.2 performed at 30% R.H. and using 10% remaining charge as the cut-off level, such as at least about any of the following: 1, 1.2, 1.4, 1.6, 1.8, and 2 seconds and/or at most about any of the following: 3, 2.8, 2.6, 2.4, 2.2 and 2 seconds.

In accordance with certain embodiments of the invention, the fabric has a hydrohead from about 60 mbar to about 100 mbar, such as from at least about any of the following: 60, 65, 70, 75, and 80 mbar and/or at most about any of the following: 100, 95, 90, 85, and 80 mbar.

In accordance with certain embodiments of the invention, the fabric has a bonded area defined by a plurality of discrete bonding sites. For example, the bonded area comprises no more than 40%, no more than 30%, no more than 25%, no more than 20%, no more than 15%, no more than 10%, no more than 5%, no more than 3%.

In accordance with certain embodiments of the invention, the fabric may be provided in the form or at least as a component for a wearable article, such as a gown, a drape, a pair of pant, a jacket, or a shoe cover. As noted above, the fabric may comprise one or more discrete treated areas comprise. These one or more discrete treated areas may comprise high risk areas for exposure to bodily fluids, such as blood.

In another aspect, the present invention provides a method of forming a fabric, such as those described and disclosed herein. The method may comprise providing a fibrous substrate including a first outermost surface and a second outermost surface and digitally printing or spraying an alcohol repellent composition on at least a portion of a first outermost surface of the fabric, at least a portion of a second outermost surface of the fabric, or both. The method may also optionally comprise applying an antistatic composition on at least a portion of a first outermost surface of the fabric, at least a portion of a second outermost surface of the fabric, or both. The method may also comprise curing the alcohol repellent composition to provide an alcohol repellent fabric. In accordance with certain embodiments of the invention, the step of curing the alcohol repellent composition comprises subjecting the alcohol repellent composition to a plasma treatment (e.g., atmospheric plasma treatment). In accordance with certain embodiments of the invention, the step of curing the alcohol repellent composition is devoid of subjecting the alcohol repellent composition to an elevated temperature during the curing operation (e.g., a room temperature or above about 25° C.).

In accordance with certain embodiments of the invention, wherein providing the fibrous substrate comprises providing a pre-treated fibrous substrate. For example, the pre-treated substrate may comprise an antistatic composition located on at least a portion of the first outermost surface, on at least a portion of the second outermost surface, or both.

In accordance with certain embodiments of the invention, the method may comprise (as noted above) digitally printing or spraying an alcohol repellent composition in one or more discrete treated areas on the first outermost surface, the second outermost surface, or both.

In accordance with certain embodiments of the invention, the method may comprise bonding the fibrous structure, such as by a thermal bonding process or ultrasonic bonding process. For example, the bonding operation may comprise thermal calendering of the fabric or through-air-bonding.

### EXAMPLES

The present disclosure is further illustrated by the following examples, which in no way should be construed as being limiting. That is, the specific features described in the following examples are merely illustrative and not limiting.

A. Test Methods

Basis weight of the following examples was measured according to ASTM test method D3776. The results were provided in units of mass per unit area in g/m² (gsm).

Alcohol repellency of the following examples was measured according to test method IST 80.8

Static decay of the following examples was measured according to standard test method IST 40.2 performed at 50% RH and 30% RH using 10% remaining charge as cut-off level. A lower value indicates a short static dissipation time.

Hydrohead of the following examples was measured according to standard test method IST 80.8 and ramping up the pressure at a rate of 60 mbar/min. A larger hydrohead value is more desirable for increased the barrier performance.

Air Permeability is a measure of air flow passing through a sheet under at a stated pressure differential between the surfaces of the sheet and was conducted according to ASTM D 737, Test area 38 cm², Test Pressure @ 125 Pa, and is reported in ml/dm²/min. A larger air permeability value is indicative of improved comfort for surgical gown and drape applications.

B. General Manufacturing Method

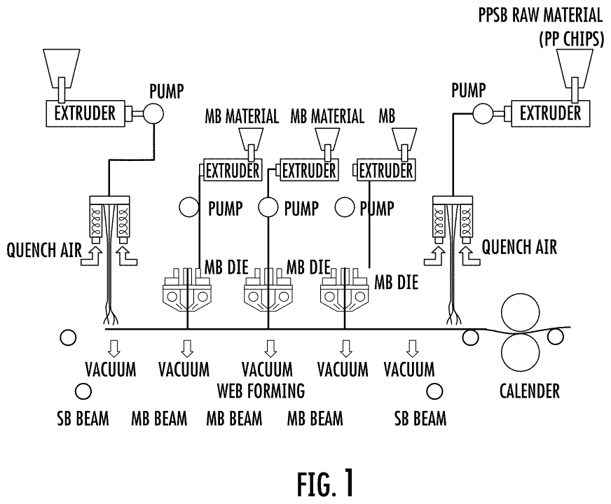

All base nonwoven samples were made from a polypropylene SMS-type nonwoven comprising at least a layer of polypropylene meltblown fibers positioned between at least two layers of polypropylene continuous spunbond filaments and point bonded using a thermal calendering operation. More specifically, the inventive sample base materials (Inventive Example A, Inventive Example B, and Inventive Example C) and Comparative Sample 1 each had a SMMMS structure made on the same a 5 beams production line known as a Reicofil 4 at similar speed and process conditions. The process consisted of first spinning continuous filaments that are deposed on a foraminous moving surface on beam 1. Beams 2, 3, and 4 provide three layers of polypropylene meltblown fibers that were deposited on top of the layer of continuous filaments provided by beam 1. Next, beam 5 was used to spin continuous filaments that were deposited on top of the meltblown fibers from beams 2, 3, and 4 to provide a composite web. The composite web was then fed to a nip point of a calender where the composite web was point bonded under a pressure of 950 N/cm at 160° C. The bonding pattern occupied about 18% of the nonwoven surface. FIG. 1 illustrates a schematic for forming SMMMS fabrics according to certain embodiments of the invention.

Comparative Sample 1

Comparative Sample 1 was a commercial blue surgical gown & drapes code SAD7006F from Berry Global and had a total basis weight of 44 gsm. The first layer of continuous filaments for the samples was about 16.8 gsm, the 3 melt-blown layers in total amounted to about 10.5 gsm, and the second layer of continuous filaments deposited was about 16.8 gsm. The bonding pattern occupied about 18% of the nonwoven surface and point bonded as noted above. This sample material was made from the traditional topical padding methods dip/nip with can and oven drying/curing of an alcohol repellent and an antistatic finish.

In particular, the sample material (e.g., bonded fabric) was processed on a finishing line where a solution which was made of deionized water containing C6 fluorochemical emulsion, antistatic agent, and wetting agent was padded onto the fabric. The excess solution was removed to achieve a wet content of about 60% (e.g., the solution remaining in the fabric weighted 60% of the weight of the dry fabric) and the fabric was dried using banks of steam cans. For this Comparative Sample 1 a fabric surface temperature of 120° C. was attained during drying. Total % Fluorine on fabric was around 0.167% tested by Burning method reference ASTM D240.

This treated Comparative Sample 1 was tested for hydrohead using the standard test method IST 80.6 and ramping the pressure at a rate of 60 mbar/min. Comparative Sample 1 was also tested for alcohol repellency using the test method IST 80.8 and the static decay was tested using the standard test method IST 40.2 performed at 30% RH using 10% remaining charge as cut-off level. The properties of this resulting fabric (i.e., Comparative Sample 1) are summarized in Table 2 below. For this type of barrier fabric, the alcohol repellency needs to be at 7 or better, and a common target is 8. A static decay at 30% RH with 10% cut-off needs to achieve a target of 9 seconds and less than 30 seconds. An accelerated aging test for Comparative Sample 1 (e.g., at 45.6 days that was equivalent to 12 months) was performed by using aging conditions listed in Table 1, in which ASTM F 1980-02 was used as reference. The temperature and humidity controlled chamber was set at 55° C. and 75% RH. After 45.6 days aging, the physical property for this aged nonwoven (i.e., Comparative Sample 1-1) were tested. Results are provided in Table 2.

TABLE 1
Accelerate Aging Test Conditions

| | | | Days to Age | Days to Age | Days to Age | Days to Age |
|---|---|---|---|---|---|---|
| Aging Temp | Aging RH | Ambient Temp | 1 month at | 3 month at | 6 month at | 12 month at |
| (° C.) | (%) | (° C.) | Ambient | Ambient | Ambient | Ambient |
| 55 | 75 | 25 | 3.8 | 11.4 | 22.8 | 45.6 |

### Inventive Example A

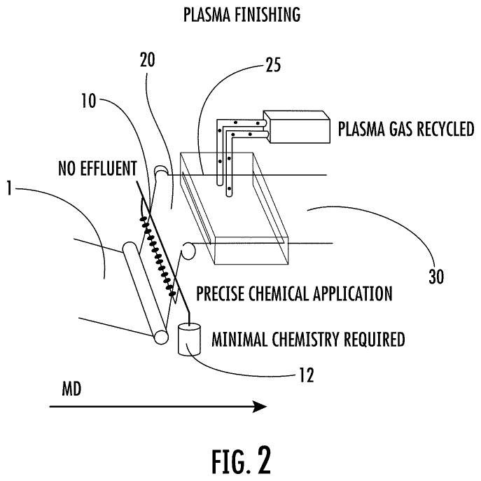

Inventive Example A used a similar starting material as Comparative Example 1. That is, the starting material was a commercial blue surgical gown & drapes code SAD7006F from Berry Global having a SMMMS structure with a total basis weight of 44 gsm. The SMMMS nonwoven was pre-coated with around 0.34% by weight add-on of an antistatic agent onto the smooth side of nonwoven via a kiss roller and dried by dryer. Subsequently, a C6 fluorine chemical formulation was sprayed via an APJeT® apparatus onto the embossing side (i.e., the side opposite of the antistatic agent). The C6 fluorine chemical formulation was then subjected to a plasma treatment (while on the embossing side of nonwoven) to cure the C6 fluorochemical at a rate of 10 meter/min speed. The total % Fluorine on fabric was around 0.277% tested by Burning method reference ASTM D240. FIG. 2 represents a schematic of the coating and curing operation. For instance, FIG. 2 shows the SMMMS nonwoven 1 traveling in the machine direction (MD). The SMMMS nonwoven 1 is first coated via an APJeT® apparatus 10 that digitally prints or sprays the C6 fluorochemical formulation onto the SMMMS nonwoven. The APJeT® apparatus 10 is fed by a supply container 12 that includes the C6 fluorochemical formulation. The C6-coated SMMMS nonwoven 20 travels from the APJeT® apparatus 10 to an atmospheric plasma curing operation 25 where the C6 fluorochemical formulation is cured. The C6-coated and cured SMMMS nonwoven 30 may then be supplied to a roll.

This treated Inventive Example A was tested for hydrohead using the standard test method IST 80.6 and ramping the pressure at a rate of 60 mbar/min. The sample was also tested for alcohol repellency using the test method IST 80.8 and the static decay was tested using the standard test method IST 40.2 performed at 50% RH and 30% RH using 10% remaining charge as cut-off level. The properties of this resulting fabric are summarized in Table 2 below. For this type of barrier fabric the alcohol repellency needs to be at 7 or better, a common target is 8. A static decay at 30% RH with 10% cut-off needs to target 9 seconds and less than 30 seconds. A static decay at 50% RH with 10% cut-off needs to be less than 0.5 second.

As this Inventive Example A material will be used as a medical surgical gown and drape, this material will go through an Ethylene Oxide (known as EtO) Sterilization process prior to being provided to the end user hospitals. In order to validate Inventive Example A material as suitable for use as a surgical gown, Inventive Example A was converted into surgical gown. After conversion into surgical gowns noted as Inventive Example A-1, the physical properties of this resulting fabric (i.e., Inventive Example A-1) were tested and the results are summarized in Table 2 below. For EtO sterilization, the validated EtO sterilization method according to ISO 11135-1 was used to sterilize Inventive Example A-1 surgical gown samples to provide sterilized samples (i.e., Inventive Example A-2). After sterilization, the physical properties of Inventive Example A-2 were tested and are summarized in Table 2 below. An accelerate aging test for Inventive Example A-2 at 45.6 days equivalent to 1 year (12 months) using aging conditions listed in Table 1 was also performed with ASTM F 1980-02 as reference. The temperature and humidity controlled chamber was set at 55° C. and 75% RH. After 45.6 days, the physical property for the resulting aged sample (i.e., Inventive Example A-3) were tested and results are provided in Table 2.

### Inventive Example B

Inventive Example B used a similar starting material as Comparative Example 1 and Inventive Example A. That is, the starting material was a commercial blue surgical gown & drapes code SAD7006F from Berry Global having a SMMMS structure with a total basis weight of 44 gsm. The SMMMS nonwoven was pre-coated with around 0.34% by weight add-on of an antistatic agent onto the smooth side of nonwoven via a kiss roller and dried by dryer. Subsequently, a C6 fluorine chemical formulation was sprayed via an APJeT® apparatus onto the embossing side (i.e., the side opposite of the antistatic agent). The C6 fluorine chemical formulation was then subjected to a plasma treatment (while on the embossing side of nonwoven) to cure the C6 fluorochemical at a rate of 300 meter/min speed. The total % Fluorine on fabric was around 0.258% tested by Burning method reference ASTM D240.

Inventive Example B was tested for hydrohead using the standard test method IST 80.6 and ramping the pressure at a rate of 60 mbar/min. The sample was also tested for alcohol repellency using the test method IST 80.8 and the static decay was tested using the standard test method IST 40.2 performed at 50% Rh and 30% RH using 10% remaining charge as cut-off level. The properties of this resulting fabric are summarized in Table 3 below. For this type of barrier fabric the alcohol repellency needs to be at 7 or better and, a common target is 8. A static decay at 30% RH with 10% cut-off needs to target 9 seconds or less than 30 seconds. A static decay at 50% RH with 10% cut-off need to be less than 0.5 second to meet medical surgical and drapes requirement.

TABLE 2
Test Result Data Summary

| | | | | | | | | | IST | IST |
|---|---|---|---|---|---|---|---|---|---|---|
| | | | | | | | | | 40.2 | 40.2 |
| | ASTM | ASTM | ASTM | ASTM | ASTM | | | IST | CD Static | CD Static |
| | D3776 | D5034 | D5034 | D5587 | D5587 | ASTM | IST | 80.8 | Decay (50% | Decay (30% |
| | Full | MD Grab | CD Grab | MD Trap | CD Trap | D737 | 80.6 | Alcohol | RH/10% | RH/10% |
| | Weight | Tensile | Tensile | Tear | Tear | Air Perm | HSH | Repellency | Cut-Off) | Cut-Off) |
| Sample ID | g/m² | lb | lb | lb | lb | cfm | mbar | rate | sec | sec |
| Inventive Example A | 44.35 | 21.18 | 13.89 | 4.37 | 8.28 | 35.0 | 60.0 | 8 | 0.10 | 2.26 |
| nonwoven | | | | | | | | | | |
| Inventive Example A-1 | 44.30 | 26.16 | 14.96 | 4.36 | 8.14 | 37.6 | 61.2 | 8 | 0.02 | 3.13 |
| nonwoven Converted | | | | | | | | | | |
| Surgical Gown | | | | | | | | | | |
| Inventive Example A-2 | 45.06 | 25.45 | 15.19 | 4.36 | 7.89 | 39.4 | 60.8 | 8 | 0.01 | 1.26 |
| nonwoven Converted | | | | | | | | | | |
| Surgical Gown with | | | | | | | | | | |
| EtO Sterilization | | | | | | | | | | |
| Inventive Example A-3 | 44.13 | 23.14 | 13.12 | 3.66 | 8.01 | 44.4 | 60.7 | 8 | 0.02 | 2.00 |
| nonwoven Converted | | | | | | | | | | |
| Surgical Gown with EtO | | | | | | | | | | |
| Sterilization | | | | | | | | | | |
| (After 1 year | | | | | | | | | | |
| Accelerate Aging) | | | | | | | | | | |
| Comparative Sample 1 | 43.14 | | 13.35 | 3.01 | | 42.40 | 57.2 | 8 | | 7.42 |
| nonwoven | | | | | | | | | | |
| Comparative Sample 1-1 | 43.40 | | 11.93 | 2.95 | | 41.80 | 56.4 | 8 | | 13.08 |
| nonwoven | | | | | | | | | | |
| (After 1 year | | | | | | | | | | |
| Accelerate Aging) | | | | | | | | | | |

TABLE 3
Test Result Data Summary for Inventive Example B

| | | | | | | | IST | IST |
|---|---|---|---|---|---|---|---|---|
| | | | | | | | 40.2 | 40.2 |
| | ASTM | ASTM | ASTM | | | IST | CD Static | CD Static |
| | D3776 | D5034 | D5034 | ASTM | IST | 80.8 | Decay (50% | Decay (30% |
| | Full | MD Grab | CD Grab | D737 | 80.6 | Alcohol | RH/10% | RH/10% |
| | Weight | Tensile | Tensile | Air Perm | HSH | Repellency | Cut-Off) | Cut-Off) |
| Sample ID | g/m² | lb | lb | cfm | mbar | rate | sec | sec |
| Inventive Example B | 45.03 | 24.17 | 14.45 | 43.5 | 61.0 | 7.33 | 0.29 | 8.50 |
| nonwoven | | | | | | | | |

From a comparison of the data in Table 2, APJeT® Plasma dry technology coated C6 fluorochemical material of Inventive Example A had a similar performance to current commercially validated blue surgical gown & drapes material of Comparative Sample 1. The hydrohead, alcohol repellency rating, and anti-static (Static Decay) data are similar between the two materials despite the two materials being treated by a different coating process. Interestingly, Inventive Example A had an improved grab tensile strength. For instance, the MD trap tear had increased about 45% by use of the APJeT® Plasma dry technology process. Additionally, after the APJeT® Plasma dry technology coated C6 fluorochemical material (i.e., Inventive Example A) was converted to surgical gown and subjected to EtO sterilization, the physical properties were retained.

From comparison of the one year accelerate aging test data for Inventive Example A-3 (e.g., produced with the APJeT® Plasma dry technology) and the Comparative Sample 1-1, Example A-3 beneficially had less degradation.

The EtO sterilized surgical gown (i.e., Inventive Example A-2) had also been tested for Cytotoxicity ISO MEM Elution according to ISO 10993-5 Using L-929 Cells and RIPT Skin Irritation Test (Repeated Insult Patch Test) according to ISO 10993-10. Both test result passed and meet medical device surgical gown requirement.

From the data shown in Table 3, Inventive Example B achieved similar physical properties and performance to the commercially validated blue surgical gown & drape material of Comparative Sample 1.

### Inventive Example C

Inventive Example C used a similar starting material to Inventive Example B. That is, the starting material was a commercial blue surgical gown & drapes code SAD7088B from Berry Global having a SMMMS structure with a total basis weight of 44 gsm. The SMMMS nonwoven was coated/printed with a pre-mixed formulation including (i) 0.4% by weight add-on (C-1) or 0.5% by weight add-on (C-2) of an antistatic agent, and (ii) a C6 fluorochemical via an APJeT® apparatus onto the embossing side in one step. The antistatic agent and C6 fluorochemical pre-mixed formulation that was coated on the embossed side was then subjected to a plasma treatment (while on the embossing side of nonwoven) to cure the C6 fluorochemical at a rate of 300 meter/min speed. The total % Fluorine on fabric is similar to Inventive Example B.

Inventive Examples C, C-1, and C-2 were tested for hydrohead using the standard test method IST 80.6 and ramping the pressure at a rate of 60 mbar/min. The samples were also tested for alcohol repellency using the test method IST 80.8 and the static decay was tested using the standard test method IST 40.2 performed at 30% RH using 10% remaining charge as cut-off level. The properties of this resulting fabric are summarized in Table 4 below. For this type of barrier fabric the alcohol repellency needs to be at 7 or better and, a common target is 8. A static decay at 30% RH with 10% cut-off needs to target 9 seconds or less than 30 seconds. A static decay at 50% RH with 10% cut-off need to be less than 0.5 second to meet medical surgical and drapes requirement.

TABLE 4
Test Result Data Summary for Inventive Examples C, C-1, and C-2

| | | IST 80.8 | 30% RH/10% Cut-Off | |
|---|---|---|---|---|
| | IST 80.6 | Alcohol | IST 40.2 | |
| | HSH | Repellency | MD Static | CD Static |
| | (Face) | (Face/Blue) | Decay | Decay |
| Sample ID | mbar | rate | sec | sec |
| Inventive Example C | 53.5 | 1 | NC | NC |
| (Untreated base nonwoven) | | | | |
| Inventive Example C-1 | 52.5 | 7.5 | 0.18 | 0.44 |
| (AR/AS coated with 0.4% AS) | | | | |
| Inventive Example C-2 | 47.5 | 7 | 0.07 | 0.26 |
| (AR/AS coated with 0.5% AS) | | | | |

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A fabric, comprising:
(i) a fibrous substrate including a first outermost surface and a second outermost surface;
(ii) a digitally printed or sprayed alcohol repellent composition located on at least a portion of a first outermost surface of the fabric, at least a portion of a second outermost surface of the fabric, or both; and
(iii) an antistatic composition located on at least a portion of a first outermost surface of the fabric, at least a portion of a second outermost surface of the fabric, or both.

2. The fabric of claim 1, wherein the digitally printed or sprayed alcohol repellent composition comprises a plasma cured composition including at least one fluorochemical comprising at least one C4 fluorochemical, a C6 fluorochemical, a C8 fluorochemical, a C10 fluorochemical, or any combination thereof.

3. The fabric of claim 1, wherein digitally printed or sprayed alcohol repellent composition comprises a plasma cured composition; wherein the plasma cured composition comprises a polymerized composition of a free-radically curable monomeric composition including at least a first group of monomers including at least one free-radically polymerizable functional group and at least one side chain comprising from about 4 to about 10 fluorinated carbon atoms.

4. The fabric of claim 1, wherein the digitally printed or sprayed alcohol repellent composition comprises a plasma cured composition; wherein the plasma cured composition comprises a polymerized composition including a perfluoroalkylethylmethacrylate containing at least one side chain from about 4 to about 10 fluorinated carbon atoms.

5. The fabric of claim 1, wherein the digitally printed or sprayed alcohol repellent composition comprises from about 0.01 to about 10% by weight of the fabric.

6. The fabric of claim 5, wherein the fabric comprises from about 0.01 to about 1% by weight of fluorine.

7. The fabric of claim 1, wherein the antistatic composition comprises from about 0.01 to about 10% by weight of the fabric.

8. The fabric of claim 1, wherein the fabric has a first ratio between the digitally printed or sprayed alcohol repellent composition (% weight of the fabric on a dry basis) to the antistatic composition (% weight of the fabric on a dry basis) from about 0.2:1 to about 3:1.

9. The fabric of claim 1, wherein the fabric has a second ratio between the fluorine content attributed to the digitally printed or sprayed alcohol repellent composition (% weight of the fabric on a dry basis) to the antistatic agent (% weight of the fabric on a dry basis) from about 0.2:1 to about 3:1.

10. The fabric of claim 1, wherein the first outermost surface, the second outermost surface, or both comprise a blended coating in cured form including the digitally printed or sprayed alcohol repellent composition and that antistatic composition.

11. The fabric of claim 1, wherein the fibrous substrate comprises one or more spunbond layers, one or more meltblown layers, one or more melt-fibrillated layers, one or more electrospun layers, one or more carded nonwoven layers, one or more hydroentangled layers, or any combinations thereof.

12. The fabric of claim 1, wherein the fibrous structure further comprises one or more layers containing a plurality of cellulosic fibers; wherein the plurality of cellulosic fibers comprises a plurality of natural synthetic fibers, a plurality of synthetic cellulosic fibers, or combinations thereof.

13. The fabric of claim 1, wherein the fabric further comprises at least one binder.

14. The fabric of claim 1, wherein the fabric has one or more of the following: (i) an alcohol repellency rating of at least 7 as determined according to IST 80.8; (ii) a static decay of from about 0.01 to about 0.5 seconds as tested according to IST 40.2 performed at 50% R.H. and using 10% remaining charge as the cut-off level; (iii) a static decay of from about 1 to about 3 seconds as tested according to IST 40.2 performed at 30% R.H. and using 10% remaining charge as the cut-off level; and (iv) a hydrohead from about 60 mbar to about 100 mbar.

15. The fabric of claim 1, wherein the digitally printed or sprayed alcohol repellent composition located on at least the first outermost surface is located in one or more discrete treated areas, and wherein the one or more discrete treated areas cover from about 1% to about 99% of the first outermost surface.

16. A method of forming a fabric, comprising:
(i) providing a fibrous substrate including a first outermost surface and a second outermost surface;
(ii) digitally printing or spraying an alcohol repellent composition on at least a portion of at least the first outermost surface;
(iii) optionally applying an antistatic composition on at least a portion of the second outermost surface; and
(iv) curing the alcohol repellent composition to provide the fabric.

17. The method of claim 16, wherein curing the alcohol repellent composition comprises subjecting the alcohol repellent composition to a plasma treatment.

18. The method of claim 17, wherein the plasma treatment comprises an atmospheric pressure plasma treatment.

19. The method of claim 16, wherein curing the alcohol repellent composition is devoid of subjecting the alcohol repellent composition to an elevated temperature during the curing operation.

20. The method of claim 16, wherein digitally printing or spraying an alcohol repellent composition comprises applying the alcohol repellent composition in one or more discrete treated areas.
